(19) United States
(12) Patent Application Publication
(10) Pub. No.: US 2008/0194029 A1
(43) Pub. Date: Aug. 14, 2008

(54) Method for Increasing the Ratio of Homologous to Non-Homologous Recombination

(76) Inventors: Peter Hegemann, Barbing (DE); Markus Fuhrmann, Teublitz (DE)

Correspondence Address: CLARK & ELBING LLP, 101 FEDERAL STREET, BOSTON, MA, 02110, US

(21) Appl. No.: 11/579,787

(22) PCT Filed: May 9, 2005
(86) PCT No.: PCT/EP2005/005008

§ 371 Date: Mar. 9, 2007

(30) Foreign Application Priority Data
May 7, 2004 (EP) 04010957.1

(51) Int. Cl. C12N 15/82; C12N 5/02

(52) U.S. Cl. 435/468; 435/419; 435/440

(57) ABSTRACT

Gene targeting allows the deletion (knock out), the repair (rescuing) and the modification (gene mutation) of a selected gene and the functional analysis of any gene of interest. Targeting of nuclear genes has been a very inefficient process in most eukaryotes including plants and animals due to the dominance of illegitimate integration of the applied DNA into non-homologous regions of the genome. The present invention provides a method for increasing the ratio of homologous to non-homologous recombination of a polynucleotide into a host cell's DNA by suppressing non-homologous recombination. Surprisingly, the number of non-homologous recombination events can be reduced if the polynucleotide is applied as a purified single-stranded DNA, preferably coated with a single strand binding protein.

The present invention relates to a method for increasing the ratio of homologous to non-homologous recombination of a polypeptide into a host cell's DNA and to a mixture of transformants obtainable by said process.

### BACKGROUND OF THE INVENTION

Targeted gene disruption or modification allows the introduction of in vitro generated mutations, including null mutations, into the genome of a model organism but also can be used for rescuing genes with an abnormal function. A modification of gene function can also be achieved by application of antisense technologies, but in this case silencing is only partial and temporary, may strongly depend on the physiological conditions and cannot be specifically applied to a gene to which related genes in the genome exist.

The successful application of targeted gene disruption is dependent on the ratio of homologous recombination (HR, FIG. 1) to illegitimate non-homologous integration (NHI, FIG. 2) events (HR/NHI) during nuclear transformation. This ratio is extremely variable among different eukaryotes. Several lower eukaryotes such as yeasts, some filamentous fungi, Trypanosomatideae and the moss Physcomitrella patens (a plant with a predominance of the haplophase in the life cycle; Schaefer and Zryd 1995, Plant J. 11,1195-1206 and literature therein) show a HR/NHI ratio above 10%. In archaea, in many lower eukaryotes like algae and especially in most higher eukaryotes the HR/NHI ratio is very low. It varies between 10⁻² and 10⁻³ in animal cells (Bollag et al., 1989 Annu. Rev. Genet 23, 199-225) and between 10⁻³ and 10⁻⁶ in plant cells (Miao and Lam, 1995 Plant. J., 7, 359-365). All these numbers are based on experiments, in which double stranded DNA (dsDNA) has been used as gene targeting substrate.

Other disadvantages that correspond to NHI in genetic transformation include the unpredictable disruption of host genes by the integrating DNA and unpredictable positional effects caused by the random integration of transforming DNA into chromatin regions of different transcriptional activity and accessibility.

Several approaches for identifying, selecting and enriching homologous recombination events have been developed for plants, mammalian cells and archaea. They involve the application of two marker genes, one for positive selection and another one outside a homologous region for suppression of the non-homologous integration, called negative selection marker (FIG. 1). The most promising negative selection marker in plants still is the diphtheria-toxin-A gene (Terada, et. al. 2002 Nature Biotechnol. 20,1030-1034). However, in rice the number of transformants generated per μg of transforming DNA is reduced only by a factor of between 10 and 100, indicating that the negative selection marker is not efficiently expressed or at least partially lost during the NHI event. Moreover, negative selection markers select for double cross over events and suppress single-cross over events, which appear to be by far more often than double cross over. Hence such markers should decrease the total number of homologous recombinants. As a consequence the resulting HR/NHI rate might become even lower using this approach. In line with this argumentation, it was not possible to achieve a targeted disruption of all plant genes tested, despite the high quantity of transformants analyzed in some cases (Thykjar et al., 1997 J. Mol. Biol., 35, 523-530).

An alternative approach to overcome the problem of the low frequency of homologous recombination in plants is to over-express well characterized heterologous or endogenous genes that encode proteins which are involved in homologous recombination (Shalev et al., 1999 Proc. Nati. Acad. Sci. 96, 7398-7402). RecA protein plays a central role in the recombination pathway of bacteria. Homologues of bacterial RecA are found in all three domains of life: prokaryotes, archaea and eukaryotes including Saccharomyces cerevisiae, Ustilago maydis, Xenopus laevis, Lilium longiflorum, Neurospora crassa, Arabidopsis thaliana, mouse, chicken, and man, suggesting that the machinery involved in recombination is highly conserved among all organisms from bacteria to man (Camerini-Otero and Hsieh, 1995 Annu. Rev. Genetics, 29: 509-532).

For tobacco protoplasts it was found that the expression of the Escherichia coli recA gene stimulated intrachromosomal recombination between rather short (only 325 bp) homologous regions 10-fold. Furthermore, repairing of mitomycin C-induced damage was three times more efficient in recA expressing cells than in wild-type cells (Reiss et al., 1996. Proc. Natl. Acad. Sci. 93, 3094-3098).

RuvC is an endonuclease involved in one of the main recombination pathways in E. coli that binds specifically to Holliday junctions, preformed by RecA, and promotes their subsequent resolution. It was shown for tobacco plants that over-expression of the nucleus-targeted ruvC gene from E. coli leads to an increase of the homologous recombination level between two co-transformed plasmids by a factor of 56 and intra-chromosomal recombination between two directly repeated homologous regions was increased 11 fold (Shalev et al., 1999 Proc. Natl. Acad. Sci. 96, 7398-7402). These data suggest that the low expression of the recA and ruvC homologs in plants might be a factor contributing to the low rates of homologous recombination in plants. All HR-stimulation experiments have been carried out with dsDNA.

Orr-Weaver et al. (1981, PNAS 78, 358-361) demonstrated that homologous recombination in yeast can be stimulated to some extent by the introduction of double stranded breaks into duplex DNA substrates. Other experiments have demonstrated that a double strand break in the chromosomal target locus enhances the frequency of localized recombination events (Cohen-Tannoudji, 1998 Mol. Cell Biol. 18, 1444 and Lit. therein). However, double strand breaks have been only discussed with respect to mechanistical considerations (Shinohara & Ogawa 1995, Trends Biol Sci. 20, 387-391) and not with respect to HR/NHI-ratios. Application of this technique to human stem cells improved the rate of gene targeting to 3%-5% of all generated recovered cell lines (Porteus & Baltimore, 2003, Science 300, 763). However, this method is only applicable in rare cases because it is difficult to find a restriction enzyme that, in a large genome, cuts with a sufficiently high specificity even if enzymes with 18 bp recognition sites are used (Bibikova et al. 2003, Science 300, 764).

Originally, it has been shown for the yeast Saccharomyces cerevisiae that dsDNA and ssDNA can be used for gene targeting almost equally well (Simon & Moore 1987, Mol Cell. Biol. 7, 2329-2334). However, these experiments did not allow any conclusion about higher eukaryotes, since experiments in yeast do not allow to monitor non-homologous gene integration (NHI); therefore, the ratio HR/NHI cannot be determined. NHI is a rare event in yeast under any conditions but is reported to be by far the most dominant process in algae, higher plants and animals (Bollag et al., 1989 Annu. Rev. Genet 23, 199-225; Miao and Lam 1995 Plant J., 7, 359-365.; Nelson and Lefebvre, 1995, Mol Cell Biol. 15, 5762-5769).

For mammalian cells, it also has been shown that ssDNA can, like dsDNA, participate in recombination processes in vivo and in a nuclear extract-catalyzed in vitro system (Rauth et al. 1986, PNAS 83, 5587-5591). But again, these authors did not determine HR/NHI ratios.

Baur et al. (1990, Mol. Cell Biol. 10, 492) and Bilang et al. (1992, Mol Cell Biol 12, 329-336) studied extra-chromosomal homologous recombination in tobacco protoplasts and found that ssDNA is an efficient substrate for recombination similar to dsDNA. In these and many later experiments specificity of gene targeting in relation to NHI was not evaluated because in general only homologous recombination between two overlapping truncated selection marker genes was tested. None of each is active by itself and they can only provide resistance after homologous recombination. The problem of the low ratio between HR/NHI is not solved (Bouche & Bouchez 2001, Curr. Opin. Plant Biol. 4,111-117, Terada et al. 2004, Plant Cell Reports, 22, 653-659).

A very popular method for introducing foreign DNA into a plant host is the application of plant infecting Agrobacteria. The transfer of Agrobacterium T-DNA to plant cells involves the induction of Ti plasmid virulescence genes. This induction results in the generation of linear single stranded copies of the T-DNA which are thought to be transferred to the plant cell. A central requirement of this ssDNA transfer model is that the plant cell immediately generates a second strand and integrates the resulting dsDNA into its genome. This integration normally occurs randomly, probably because dsDNA is the active species. Furner et al. (1989, Mol. Gen. Genet. 220, 65-68) incubated plant protoplasts with ssDNA and dsDNA and found that the transformation efficiency is similar. The authors concluded that the introduced DNA becomes double stranded before it is integrated.

Recently, Adeno-associated virus vectors (AAV) have been used to achieve HR in human somatic cells (Hirata et al. 2002, Nat. Biotechnol. 20,735-738). The combination with double stranded breaks (DSB) again made this technique more efficient Absolute gene targeting frequencies reach 1% with a dual vector system in which one recombinant AAV (rAAV) provides a gene targeting substrate and a second vector expresses the nuclease that creates a DSB in the target gene (Miller et al. 2003 Mol. Cell Biol. 23, 3550-3557 and Porteus et al. 2003 Mol. Cell Biol. 23, 3558-3565). The major advantage of the AAV method is the efficient delivery of DNA into human cells rather than a high ratio of HR/NHI for use in gene therapy. But, this method is also limited since the DNA-insert must not exceed 4.7 kb (Smith 1995, Ann. Rev Microbiol. 49, 807-838) and, second, the host range is very narrow, which means that this system cannot be transferred to plant systems or any prokaryote.

The U.S. Pat. No. 6,271,360 and U.S. Pat. No. 6,479,292 disclose the use of short single stranded oligonucleotides (up to 55 or 65 nucleotides in length) for introducing small changes into different target genomes. The main disadvantage is that the method is intrinsically limited to the application in changes that result in a directly selectable phenotype. First, because the reported ratio between the introduction of the vector into the cell and the resulting targeting events is in the range of only 10⁻³. Second, because this method is limited to introducing only very small changes, usually on single or few nucleotides at the region of homology such that larger sequences, e.g. marker genes, cannot be introduced at the desired site of the genome by this approach. Thus, a direct selection by a marker gene is not possible due to the size limitation of the ss oligonucleotides. Not even one of the shortest selectable marker genes as it is the zeocin resistance gene ble from Streptoalloteichus hindustanus with a length of 375 bp in the coding region can be included in such oligonucleotides. in contrast, longer sequences allow the introduction of larger marker genes, non-selectable reporters and structural genes. Additionally, multiple gene disruptions become feasible to generate several knockouts per cell line. Thus, the targeting of genes for creating non-selectable null-mutations is unfeasible using the oligonucleotide approach.

An ssDNA fragment of 488 bp has been applied to induce specific genetic changes in the cystic fibrosis transmembrane conductance regulator gene (Gonċz et al.,1998, Hum. Mol. Genetics 7, 1913; Kunzelmann et al., 1996, Gene Ther. 3, 859-67). The common feature of these approaches is the lack of a selectable marker gene inside the region of homology that could be used for selection of gene-targeting events, resulting in null-mutations of the respective gene locus. This limitation is most likely a consequence of the limited length of the ssDNA species used in all these experiments.

Green microalgae are of great value, both as organisms for fundamental biological research and as a resource for the biotechnological industry. The potential of the green unicellular alga Chlamydomomas reinhardtii is especially promising because this unicellular eukaryote, also called the green yeast (Rochaix 1995 Annu. Rev. Genet. 29, 209-230), represents a powerful model system for studying cell and molecular biology of photosynthetic eukaryotes. C. reinhardtii is capable of photoautotrophic growth on pure mineral medium and can be readily cultured in large quantities and to high cell densities even in the absence of light. Because of its well-defined genetics C. reinhardtii is an ideal system for studying photosynthesis, chloroplast biogenesis, flagella function, phototaxis etc. The value of this organism has been greatly increased during recent years by the development of efficient methods for nuclear, chloroplast and mitochondrial transformation (Lumbreras & Purton, 1998, Protist 149, 23-27).

Nuclear transformants have been obtained using intact and chimeric C. reinhardtii genes as selection markers, which complement auxotrophic mutations (Kindle 1990, PNAS 87,1228-1232; Purton & Rochaix 1995, Eur. J. Phycol. 30,141-148). However, genetic and molecular analyses of nuclear transformants reveal that integration of the DNA predominantly occurs via non-homologous recombination resulting in the introduction of the marker-DNA at apparently random loci (Debuchy et al. 1989, EMBO J. 8,2803-2809). Further, application of C. reinhardtii as a model system and for technical use urgently demands techniques for targeted gene disruption and gene replacement enabling the study of gene functions.

Ongoing genome projects offer the scientific community a wealth of information concerning sequence and organization of the C. reinhardtii genome. Generation of 200,000 Chlamydomons cDNA sequences has allowed the fast identification of thousands of genes with homology to genes already known from other organisms (http://www.biology.duke.edu/chlamy_genome/) and many other “new” genes of potential interest. Microarrays with all plastid genes and 3,000 nuclear genes are available. The complete chloroplast genome and a rough draft of the near complete genome sequence was made publicly accessible in the early part of 2003. This sequence has been partially annotated and both cDNA information and molecular markers have been anchored to the sequence (Grossman et al. 2003). These advances have dramatically enhanced the utility of C. reinhardtii as a model system. However, to fully exploit the information for the understanding of the different gene products, targeted disruption of selected genes is more necessary than ever before.

Earlier experiments studying recombination in C. reinhardtii indicated that the machinery for homologous recombination exists in vegetative cells and suggested that a targeted gene disruption technique could be developed (Sodeinde & Kindle 1993, PNAS 90, 9199-9203; Gumpel et al. 1994, Curr. Genet 26;438-442). Using the efficient endogenous marker genes nit1 and arg7 the authors have shown that homologous recombination between two co-transforming non-functional gene copies containing non-overlapping mutations occurred at a high frequency to obtain the repaired active gene. The transformation rate of such plasmid pairs reached 10-20% in comparison to the use of single plasmids with intact genes and was dependent on the length of homologous regions. A region of homology of less than 300 bp was sufficient to achieve significant HR between the plasmid pairs. The rate of transformation increased when the length of the homologous regions reached 1000 bp up to 20%. Longer regions of homology (5000 bp) led to an only marginal further stimulation up to 21%. Moreover, homologous recombination and repair was found to occur between the introduced and endogenous mutated gene copies but at a rate in a few orders of magnitude lower than the rate of extra-chromosomal recombination. For the nit1 gene the estimated ratio of homologous to non-homologous recombination events ranges between 1:40 to 1:1000 depending on transformation method used (Sodeinde and Kindle, 1993, PNAS 90, 9199-9203). Rare but detectable gene-targeted insertion was revealed at the arg7 locus (Gumpel et al. 1994, Curr. Genet 26;438-442). These rates could only be estimated by comparison to routine experiments under similar conditions. The ratio of HR/NHI could not be investigated in these experiments due to a direct selection on HR events, and counterselection against NHI. Later experiments by Nelson and Levebre (1995, Mol. Cell. Biol. 15, 5762-5769) clearly revealed that the estimates given for HR rates by Sodeinde & Kindle were by far too optimistic.

For targeted disruption of the nit8 locus these authors used the nit8 coding sequence interrupted by the cry1-1 selection marker gene that provides emetine-resistance. One of 2000 transformants selected for emetine- and chlorate-resistance (positive and negative selection) contained a homologous insertion of five copies of the disruption construct within the nit8 gene.

In view of the foregoing, there is a strong need for the development of methods improving gene targeting by increasing the ratio between homologous to non-homologous recombination. Especially in plants, the ratio between HR and NHI is extremely unfavorable.

It is therefore the goal of the present invention to provide an efficient and reliable method for increasing the ratio of homologous to non-homologous recombination by suppression of non-homologous integration of polynucleotides into the genome.

A solution to this problem is provided by the method of claim 1, allowing suppression of non-homologous recombination by the use of one or more single-stranded DNAs capable of homologous recombination with the cell's DNA. Surprisingly, the inventors observed a highly unexpected increase of the HR/NHI ratio by use of ssDNA instead of dsDNA, due to almost complete avoidance of NHI (Tab.1). Contrary to the common belief, there is no need for any single stranded DNA to be converted into a double-stranded DNA before recombination. Moreover, precaution should be taken that ssDNA is not replicated into dsDNA in the host, which again would promote random integration into the host genome. This may be achieved by preincubation of the ssDNA with specific binding proteins like SSB, recA or related proteins. Surprisingly, the inventors observed that transformation applying single stranded DNA greatly increases the ratio of HR to NHI.

In the following some of the terms used are explained further and defined in order to clarify how they should be interpreted in the context of this application.

“Homologous recombination” (HR) or “legitimate recombination”: The exchange of DNA sequences between two DNA molecules, mainly two homologous chromosomes that involves loci with complete or far-reaching base sequence identity. Homologous recombination may also occur between a chromosome or other cellular DNA and an extra-chromosomal element introduced into the cell, provided that the extracellular element carries a region with complete or nearly complete sequence complementarity.

A sequence of 14 bp (4¹⁴ possible variations) occurs only once on average in a genome of 200 Mbp. To define significant “unique” homology, a stretch of at least 16 bp should be identical between the host DNA and the recombinant targeting DNA. Longer regions of homology with at least 90% identity of all nucleotide positions of the corresponding strands might increase the probability of HR by providing a larger quantity of possible sites of HR within the DNA of interest.

“Non-homologous or illegitimate recombination”: The exchange of DNA sequences between two DNA molecules, mainly two non-homologous chromosomes. Non-homologous recombination may also occur between a chromosome or other cellular DNA and an extrachromosomal element introduced into the cell, that show no complementarity sequence.

“HR/NHI”: Ratio of homologous recombination to non-homologous integration events.

“Host cell”: Any cell that might serve as a recipient to be transformed with a recombinant polynucleotide.

“Polynucleotide”: Any DNA, RNA and derivatives thereof. Normally they are originating from natural sources but they might be generated by in vitro synthesis from chemically synthesized oligonucleotides.

“Selection marker”: a gene facilitating the selection of transformants containing a specific polynucleotide out of many non transformed cells. This may be a gene that encodes a protein catalyzing the destruction, sequestration, modification or the export of a toxin (e.g. an antibiotic). Selection markers also include genes coding for fluorescent proteins, proteins capable of producing bio- or chemiluminescence, or enzymes capable of producing coloured substances from suitable substrates. Also genes that are able to complement specific auxotrophic mutations are used as selection markers.

“Transformation”: Modification of a host cell's genome by external application of a polynucleotide, which is taken up and integrates into and modifies the host cell's genome.

“Transformant”: A cell that has undergone a transformation.

A technique is provided by the invention allowing the attainment of a strong increase in the ratio of homologous to non-homologous recombination in comparison to methods disclosed in the art.

In one preferred embodiment the isolated ssDNA is treated with endonucleases, to minimize traces of double-stranded DNA. Possible enzymes include specific restriction endonucleases, e.g. Dpnl, capable of cleaving methylated DNA exclusively. For a significant reduction of background clones resulting from dsDNA impurities, a ratio of ssDNA to dsDNA of at least 10 000 to about 100 000 is required. Consequently, the maximal amount of residual dsDNA in the ssDNA preparation should be less than 1 dsDNA molecule per about 10 000 to about 100 000 ssDNA molecules.

In another preferred embodiment residual dsDNA can be removed using exonuclease treatment with exonuclease III from E. coli as described.

Other preferred possibilities to obtain ssDNA with a very low degree of contamination with dsDNA employ a primer extension reaction followed by enzymatic treatment for removal of template DNA.

In another preferred embodiment the single-stranded DNA comprises a nucleic acid sequence corresponding to a nucleic acid sequence of the cell's DNA, but differing from it by deletion, addition or substitution of at least one nucleotide. The number of nucleotides not matching the host cell's DNA might vary with the length of the single-stranded DNA. Generally, a single-stranded DNA capable of homologous recombination with the host cell's genome will exhibit an identity of at least 90% of all nucleotides in a region of more than 16 bp of the host genome. The ssDNA molecules can include also stretches that are not homologous to the host genome (selectable marker genes) according to this definition. These regions should not be involved in the recombination process, but will be introduced into the genome together with the homologous part. Thereby gain-of-function and loss-of-function mutations can be introduced into the cell. Further modifications include the targeted integration at chromatin regions of high transcriptional activity for overexpression of selected genes, avoidance of unwanted positional effects upon integration into the genome, avoidance of random disruption of endogenous genes, knock-in-mutations by replacement of endogenous genes for recombinant variations, introduction of reversible gene disruptions by inclusion of recognition sites for specific recombinases, e.g. Cre recombinase or ΦC31 recombinase.

In a preferred embodiment the length of the ssDNA used in the methods above comprises 100 to 30 000 nucleotides. In a more preferred embodiment the length of the ssDNA comprises 200 to 5 000 nucleotides and in a still more preferred embodiment the length of the ssDNA comprises around 1 000 nucleotides. Despite longer ssDNAs (>200 bps) are more difficult to prepare (with any method used, primer extension reaction could terminate prematurely, ssDNA phages tend to lose unnecessary DNA portions, exonuclease treatment requires longer treatment with the possibility of side reactions, etc.) the use of longer ssDNAs is worth the effort since the efficiency of HR appeared to be higher compared to short ssDNA.

In a more preferred embodiment the ssDNA further comprises a nucleic acid sequence acting as a selection marker. The selection marker usually but not exclusively encodes a protein catalyzing the destruction of a toxin. Transformants can be selected by growing the transformed cells in the presence of the toxin, where non-transformed cells will not survive. Other selection markers may restore the ability of auxotrophic metabolic mutants to grow on minimal media, e.g. arginino succinate lyase or nitrate reductase. Fluorescent proteins, e.g. the green or red fluorescent proteins, flavinmononuclotide-binding proteins, phycobiliproteins, can be used in automated cell sorting systems to separate different cell populations. Luminescence producing proteins, e.g. luciferases, horse-radish peroxidase, phosphatases, can be used to directly visualize transformed cells with sensitive cameras. And enzymes capable of producing colored substances from different precursors can be used to stain transformants, e.g. chloramphenicol acetyltransferse, beta-galactosidase and beta-glucuronidase, arylsulfatase, alkaline, neutral and acidic phosphatases.

In a more preferred embodiment the selection marker codes for resistance to an antibiotic. Among the preferred resistance marker genes are ble (zeocin, phleomycin), aph7″ (hygromycin), aphVIII (paromomycin, kanamycin), Acetolactate-synthase (C.reinhardtii) mutant-K257T (sulfometuron methyl), Ppx1 (S-23142), Cry1-1 (emetine), cat (chloramphenicol), aadA (spectinomycin, streptomycin), D-aminoacid oxidase DAO1 (D-Ala vs. D-lle)

A particularly preferred embodiment is a selection marker derived from an amino-glycosidephosphotransferase gene (aph) and in the most preferred embodiment the aph gene is aph VIII from Streptomyces rimosus.

In another preferred embodiment the method is used for the generation of transformants by transforming the host cell with at least a single-stranded DNA capable of recombining with the cell's DNA.

Possible host cells include cells derived from prokaryotes or eukaryotes. Transformation methods include those known in the art, e.g. for prokaryotes and/or eukaryotes electroporation, calcium chloride, lithium acetate, polyethylene glycol, particle bombardment, vacuum infiltration, for plants particle bombardment, vacuum infiltration (tomato, Arabidopsis, rice, maize, wheat, potato, etc.), for algae electroporation, glass bead shaking, silica carbide whiskers, particle bombardment (Chlamydomonas, Chlorella, Dunaliella, Haematococcus, Codium, Ulva, Laminaria, Volvox), for Chiamydomonas reinhardtii electroporation, glass bead shaking, silica carbide whiskers, particle bombardment.

In a preferred embodiment the transformants are selected by use of the selection marker.

In another preferred embodiment the single-stranded DNA does not contain a nucleotide sequence that might serve as an origin of replication in order to avoid formation of dsDNA.

Surprisingly, the inventors observed that homologous recombination is extraordinarily efficient, when the single-stranded polynucleotide is covered with a single-stranded binding protein and transformation is carried out with the resulting DNA/protein filament. A preferred single-strand binding protein is recA from Streptomyces rimosus and/or rad51 from Chlamydomonas rheinhardtii or homologues thereof.

In another preferred embodiment the host organism belongs to a strain that over-expresses proteins that promote the recombination process. In a more preferred embodiment the over-expressed proteins are RecA and/or Rad51.

It is known that the proteins encoded by recA and rad51 support the homologous recombination in various organisms and that in plants over-expression of these proteins can lead to an increase in recombination as shown for double-stranded DNA. Surprisingly, the inventors could show that the supporting effect of recA and rad51 extends to homologous recombination using single-stranded DNA. Therefore, either a transformation of a polynucleotide together with recA and/or rad51 or a transformation of a cell, overexpressing recA and/or rad51, with ssDNA improves the ratio of HR to NHI significantly. Other related single-stranded binding proteins might also be useful in the methods described.

The ssDNA may be produced using a single-stranded DNA virus or bacteriophage, such as Enterobacteria phage M13 (Inoviridae) or a derivative thereof. Other viruses and phages that may be used include Plectrovirus Acholeplasma phage MV-L51 (Inoviridae), Enterobacteria phage ΦX174 (Microviridae), Spiromicrovirus Spiroplasma phage 4, Bdellomicrovirus Bdellovibrio phage MAC1, and Chlamydiamicrovirus Chlamydia phage 1(all Microviridae); Mastrevirus Maize streak virus, Curtovirus Beet Curly Top Virus, Begomovirus Bean Golden Mosaic Virus—Puerto Rico (all Geminiviridae), Circovirus Chicken anemia virus, Nanovirus Subterranean clover stunt virus (all circoviridae), Parvovirus Mice minute virus Erythrovirus B19 virus, Dependovirus Adeno-associated virus 2, Densovirus Junonia coenia densovirus, Iteravirus Bombyx mori densovirus, Brevidensovirus Aedes aegypti densovirus (all parvoviridae).

Another preferred embodiment is that the ssDNA is produced via primer extension from a linearized double-stranded plasmid. Such a DNA is easier and more quickly prepared (compared to preparation via a phage) but the amount is normally less and the length distribution is less homogenous than ssDNA prepared from phage.

Alternatively, ssDNA may be generated from a ds-fragment by treatment with exonuclease III from E. coli (Exo III) or any other enzyme having exonucleolytic activity. The method according to the present invention may be applied to eukaroytes, in particular to plants like tomato, arabidopsis, rice, maize, wheat, potato, etc.

In a preferred embodiment, the method is used to transform lower plants like green algae, which include Chlamydomonas reinhardtii, C. smithii, C. nivalis, C. allensworthii, Chlorella vulgaris, Chl. kessleri, Dunaliella salina, D. bardawil, D. acidophila, Haematococcus pluvialis, Codium bartletti (BAT), edule (EDU), fragile (FRA), muelleri (MUE), taylori (TAY), tenue (TEU), tomentosum TOM), sinuosa (SIN) & spp., Ulva lactuca (LAC), pertusa (PET), reticulate (RET), mirabilis, Laminaria angustata (ANG), bongardiana (BON), diabolica (DIA), digitata (DIG), groenlandica (GRO), hyperborea (HYP), japonica (JAP), longicruris (LOG), longissima (LOI), ochroleuca (OCH), octotensis (OCT), religiose (REL), saccharina (SAC), setchelli (SEC), sachinzii (SCH) & spp., Volvox carteri, Acetabularia acetabulum, major, Enteromorpha intestinalis, compressa (COP), clathrata (CLA), greviflei (GRE), intestinalis (INS), linza (LIZ), lomentaria (LOM), nitidum (NIT), prolifera (PRL) & spp. The most preferred species is Chlamydomonas reinhardtii

Examples for possible and non-limiting uses of the method include: i) disruption and/or restoration of endogenous genes and/or their regulatory DNA elements (promoters, enhancers, terminators) to induce specifically gain-of-function and loss-of-function mutations. ii) directed changes in metabolism to generate, modify or remove peptide and non-peptide secondary metabolites, e.g. pigments, vitamins, saturated and unsaturated fatty acids, antioxidants, energetic compounds (hydrogen, methane), iii) changes in amino acid composition of cellular polypeptides to increase nutritional value by enrichment of essential amino acids, iv) overexpression of selected genes, coding for e.g. plant, animal and/or human enzymes, immunoglobulins, peptides, hormones, etc. by site directive targeted integration at chromatin regions of high transcriptional activity, v) avoidance of epigenetic unwanted position effects on foreign gene expression upon ectopic integration into the genome. vi) avoidance of random disruption of endogenous genes resulting in unexpected and undesirable changes in phenotype of the transformants, vii) knock-in-mutations by replacement of endogenous genes for recombinant variations for essential genes, where a loss-of-function knock-out mutation would be lethal, viii) introduction of reversible gene disruptions by inclusion of recognition sites for specific recombinases, e.g. Cre recombinase or ΦC31 recombinase.

Another preferred embodiment is that the method is applied to prokaryotes, for example to Halobacterium salinarium and Natronobacterium pharaonis Examples for possible non-limiting uses are the generation and production of improved or modified light activated ion pumps (Bacteriorhodopsin and Halorhodopsin) or light triggered sensors (Sensory Rhodopsins), the generation of non-infective bacteria, bacteria capable of destruction of environmental toxins.

A further preferred embodiment is that the selection marker is constructed in such a way that it can be removed from the gene-targeted transformant. By removing the selection marker gene reactivation is possible. For such directed removal site-specific recombinases or restriction endonucleases with long (>16 bp) recognition sequences, e.g. “homing endonucleases” can be used.

The invention also relates to a mixture of transformants obtainable by transforming a host cell in the presence of one or more single-stranded DNAs (for example degenerated ssDNAs) capable of homologous recombination with the cell's DNA.

A preferred embodiment relates to a mixture of transformants, wherein the ratio of transformants subjected to homologous and non-homologous recombination events is larger than 1:100, A more preferred embodiment is that the ratio is larger than 1:10 and still more preferred is that the ration is larger than 1:3.

In the following the invention is illustrated in special embodiments by figures and examples.

### DESCRIPTION OF FIGURES

FIG. 1: Recombination between the transforming DNA and homologous host DNA. (Homologous recombination, HR). The transforming DNA comprises a positive selection marker (M1, grey) within the locus of interest. Single cross over within the homologous region (event 1. or 2.) leads to modification of the locus of interest due to insertion of M1. DNA-fragments of the locus of interest are found adjacent to the cross-over event. Double cross-over (1. and 2.) also results in locus modification by insertion of the selection marker M1 but no additional integration of plasmid DNA and no insertion of a second copy of the locus of interest. If a negative selection marker M2 is placed outside of the “locus of interest” on the targeting plasmid, transformation is biased to double cross over (positive and negative selection), because in case of M2 expression, the respective transformant should die. In case of transformation with linear DNA fragments homologous recombination by double cross over is thought to be the only integration mechanism.

FIG. 2: Non-homologous gene integration (NHI) occurs via double stranded DNA at locations of short homology (<10 bp, [symbol] ) between transforming DNA and host DNA that are found at many places throughout the host genome. It requires double-stranded cuts, annealing of the integration sites of the plasmid and the host DNA, followed by ligation. This process is often named “non homologous end joining, NHEJ”. In most cases integration is mediated by an “integrating enzyme” (integrase).

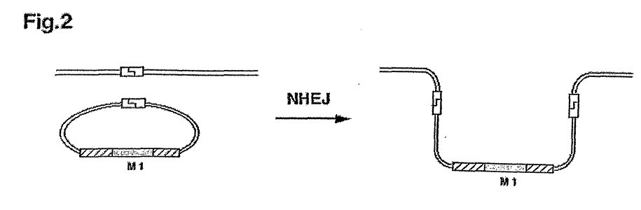

FIG. 3: Constructs that have been used for establishing directed gene targeting: GeneBank Accession Numbers of the genes used are: P=tandem promoter of hsp70/rbcS2: Accession Number AY611535; ble: Z32751; gfp: AF188479; aphVIII: AF182845, chop1: channelopsin-1: AF508967. T: terminal rbcS2 3′: X04472 dt: diphtheria toxin A: AY611535; Sequences of the constructs a) to g) are specified below. Numbers in brackets refer to the nucleotides listed under the respective Accession numbers. Additional nucleotides are indicated as G A T C.

a: P(1-507), ble(1-370), TAC, gfp (5-714), spacer, aphVIII:(1-629), spacer, rbcS2 3′ (2401-2633); the sequence is shown in SEQ ID NO: 1;

b: P(1-507), ble(1-370), TAC, gfp (5-714), spacer, aphVIII:(1-804), spacer, rbcS2 3′ (2401-2633); the sequence is shown in SEQ ID NO: 2;

c: P(1-507), aphVIII: (1-804), rbcS2 3′: (2401-2633); the sequence is shown in SEQ ID NO: 3;

d: aphVIII:(121-804), rbcS2 3′: (2401-2633); the sequence is shown in SEQ ID NO: 4;

e: P(1-1501), spacer, P(1-507), aphVIII:(1-804), rbcS2 3′ (2401-2633), spacer, P: 1-1501; the sequence is shown in SEQ ID NO: 5;

f: chop1 (262 to 3127), spacer, P: 1-507, aphVIII:(1-804), rbcS2 3′: (2401-2633), spacer, chop1(4978 to 6361), the sequence is shown in SEQ ID NO: 6;

g: chop1 (1021 to 2041), spacer:, aphVIII:(1-804), rbcS2 3′: (2401-2633), spacer, chop1 (3200 to 4580): the sequence is shown in SEQ ID NO: 7;

h: gfp(5-714), spacer, aphVIII(1-804), spacer, rbcS2 3′ (2401-2633)

### EXAMPLES

1. Development of a Detection System for Determining the Ratio of Homologous Recombination Versus Illegitimate Gene Integration

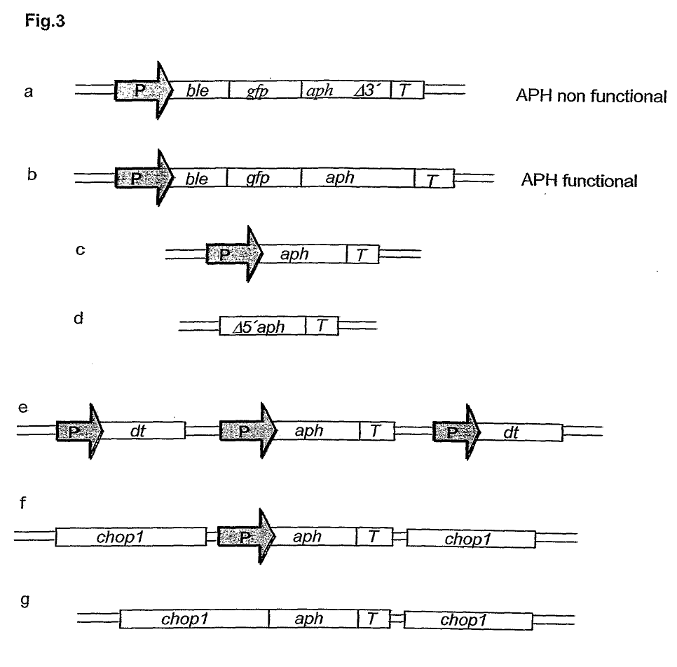

For the analysis of the efficiency of nuclear homologous recombination in relation to non-homologous gene integration a system has to be generated that discriminates HR from NHI. This is possible with a recipient Chlamydomonas reinhardtii strain (T-60), that was generated from strain cw15arg-, by insertion of a genomic DNA-element and comprising in frame a ble-gene, a gfp-gene and a 3′-truncated Δ3′-aphVIII-gene (FIG. 3a, SEQ ID NO: 1). The ble gene was used for the selection of this strain in media containing the antibiotic zeocine (derivative of phleomycine, see legend to FIG. 3) (Lumbreras et al. 1998 Plant J. 14, 441-447), Δ3′-aphVIII was used as an indicator for recombination and gfp for monitoring the expression of the fusion protein. The aphVIII gene codes for aminophosphotransferase VIII providing resistance to paromomycin.

Transformation of the Chlamydomonas reinhardtii strain CW15arg- with a functional aphVIII-marker gene containing a rbcS2-promoter and a terminator (ds-plasmid, plS103, FIG. 3c, SEQ ID NO: 3, Sizova et al., Gene 277, 221-229), resulted in 3000 clones/10 μg DNA and similar numbers were reached with the strain T-60 (Tab. 1).

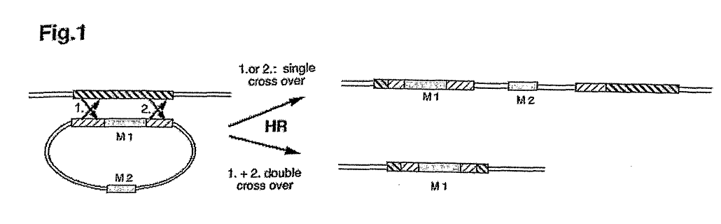

Next we have transformed Chlamydornonas with a plasmid that contained a diphtheria toxin (dt) A gene (protein sequence Accession Number: 760286A) on both sides of the aphVIII marker gene (FIG. 3e, SEQ ID NO: 5) in order to suppress illegitimate plasmid integration (negative selection, see FIG. 1). This strategy is similar to that one applied to maize (Terada et al 2002, Nat Biotechnol. 20,1030-1034). For Chlamydomonas, the dt-gene was codon-adapted by de novo gene synthesis (Fuhrmann et al. 1999, Plant J. 19, 353-361, Accession No: AY611535. Similar as in maize, the total number of clones declined by a factor of about 10 and was almost identical for both strains, CW15arg- and T-60, indicating that the principle of negative selection using the dt-gene was feasible. However, there was no indication for any dominance of homologous recombinants as shown by the fact that identical numbers of clones have been obtained for both strains.

This experiment indicates that the negative selection marker is not efficiently expressed in a lot of transformants and/or is at least partially lost during the NHI event HR/NHI ration could not be significantly enhanced using this strategy in Chlamydomonas.

To prove the frequency of homologous recombination we used a truncated ds-plasmid containing an aphVIII-gene with deletion on the 5′ part (Δ5′ aphVIII, FIG. 3d, SEQ ID NO: 4) that only generates paromomycine resistant clones after recombination with the 3′-truncated-aphVIII (Δ3′ aphVIII) of the recipient. Two transformants per 200 μg plasmid DNA (20 transformations each with 10 μg) were found in strain T60 in which the truncated Δ5′ aphVIII can undergo homologous recombination and rescue the 3′-deleted gene (Tab. 1). No transformants were found in the control strain CW15arg- (which does not carry the missing part of the aphVIII gene). Transformations with the full-length ds-aphVIII gene under identical conditions resulted in 60 000 clones because all integrations (homologous and non homologous) are resulting in active aphVIII and paromomycine resistance. Comparison of both experiments lead to the conclusion that the rate of homologous recombination was still in the range of 1 HR per 30 000 integrations (comparable to results from Nelson and Levebre 1995, Mol. Cell. Biol. 15, 5762-5769) (Tab. 1).

TABLE 1

| | CW15arg- | T60 |
|---|---|---|
| aphVIII | 3.000/3.000/0 | 3.000/3.000/0 |
| (10 μg) | | |
| dt-aphVIII-dt | 300/300/0 | 300/300/0 |
| (10 μg) | | |
| Δ5′aphVIII | 0/(60.000)*/0 | 2/(60.000)*/2 |
| (10 μg × 20 transformations) | | |
| ss(aphVIII-primer extension) | 20/20/0 | 80/nd/nd |
| (10 μg × 10 transformations) | | |
| ss(aphVIII + helper phage) | 0/0/0 | 4/3/1 |
| (10 μg × 20 transformations) | | |
| ss(aphVIII-M13 phage) | — | 30/16/4 |
| 3 μg × 10 transformations) | | |

The numbers in columns 2 and 3 mean: Total number of clones/Clones obtained by non-homologous recombination/Clones obtained by homologous recombination,
*predicted level of transformation.

2. Avoiding Non-Homologous Recombination by Using Pure Single Stranded DNA (ss-DNA)

We have transformed C. reinhardtii CW15arg- cells with a functional linear ss-aphVIII marker (plS103, FIG. 3c, SEQ ID NO. 3). Ten transformations, each with 10 μg DNA, generated only 20 transformants instead of 30 000 that had been expected from transformation with the same but double stranded marker. In the T60 recipient containing the 5′-truncated Δ5aphVIII significantly more transformants could be generated (80 instead of 20, Tab. 1). This was the first experimental indication for a significant increase of homologous recombinations events facilitated in the T60 recipient. The locus of integration has not been determined. Transformants of the strain CW15arg- could be based on non-homologous gene integration or a homologous integration into the endogenous rbcS2-promoter region. Non-homologous integrations could be caused by residual traces of dsDNA. Thus, as the next step circular ssDNA (SEQ ID NO: 3) was produced by phagemid pBlueScript II (−) and helper phage VCSM13 in M13-Phage, which should result in cleaner ssDNA compared to the formerly used polymerase reaction performed directly from the plasmid with one primer (linear PCR, primer extension). 20 transformations of CW15arg with single-stranded phage-aphVIII-DNA did not result in any transformant, whereas in the T60 recipient strain 4 transformants were generated from 20 transformations. In one of them the 3′-deletion of the recipient strains has been repaired, which led to the selective resistance against paromomycin (FIG. 1b). The repair was verified by PCR and sequencing of the aphVIII-PCR-product. It was likely that in the other transformants the plasmid integrated into homologous plasmid sequences of T60-recipient outside the aphVIII (for example within endogenous rubisco, but without a disruption of the gene, which would lead to a light-sensitive phenotype). But his has not been verified. But, in any case by use of ssDNA the HR/NHI ratio was as low as 1:3 and not 1:30 000 as found with dsDNA. In case of aphVIII the improvement was 10 000 fold.

3. Complementation of the aphVIII Gene (Gene Rescue)

The full length marker providing resistance to the antibiotic paromomycin is based on the aphVIII gene connected with a rbcs2 promoter (ribulose bisphosphate carboxylate small subunit2)/heat shock (hsp70) promoter hybrid and a rbsc2 terminator (Sizova et al. 2001), used for repairing the truncated aphVIII gene of the recipient strain T60 (FIG. 3a, SEQ ID NO: 1). Using one preferred version of the protocol ssDNA was produced via linear PCR. One primer was used per reaction. These primers were complementary to the 5′ and 3′ ends of aphIII marker. Common PCR protocols were used, i.e. primers: 5′ HSP (SEQ ID NO. 8): TGGAGCTCCACCGCGGTGG and 3′ RBCS (SEQ ID NO: 9):TGGGTACCCGCTTCAAATAC, 95° C. −5 min, 35 cycles: 95° C. 40″, 60° C. 40″, 72° C. 40″, and finally 72° C. 5 min. The total PCR product was precipitated by ETOH, and cleaved with Sac II for removal of the double-stranded template. 10 μg of the final ssDNA were used for transformation of C. reinhardtii strain CW15 cells by routine glass-bead method (Kindle 1990, Proc. Natl. Acad. Sci. USA 87, 1225-1232). The cells were in the early exponential growth phase OD_{800 nm} =0,2-0,3.

According to a second protocol version, the aphVIII marker was cloned into pKS II (−) vector (Stratagene, Amsterdam The Netherlands) that was used for the production of ssDNA by co-infection of E. coli cells with helper phage (VCSM13, Stratagene, Amsterdam The Netherlands), according to the suppliers instruction. Briefly, after 12 hours after superinfection by helper phage we centrifugate the cell culture, take the supernatant and add PEG 2000 up to 3,5% followed by precipitation by centrifugation. Then Pellet was resuspended in 0,3 M NaOAc, 1 mM EDTA followed by Phenol/Chloroform extraction. The total DNA obtained was digested with Sac II. Ds-aphVIII was removed by cleavage with Sac II. Transformation was carried out under the same as in the former protocol.

For the detection of clones with a repaired aphVIII gene and in order to discriminate them from transformants with non-homologous gene integration (NHI), integration was tested by PCR with the following primers: (Ble-fw (SEQ ID NO. 10): GAGATCGGCGAGCAGCCGTGG; Psp-Rev (SEQ ID NO: 11): GAGCAGTATCTT-CCATCCACC; AphVIIID3′-rev (SEQ ID NO: 12): ACCAGCGCGAGATCGGAGTGC) (FIG. 3). The PCR product resulting from Ble-fw and AphVIIID3′-rev primers could only appear in case of homologous recombination between the truncated and the full length copy of the aphVIII gene. The products generated by Ble-fw and Psp-rev are generated from both, repaired and nonfunctional aphVIII template, but after recombination the size of PCR product increases by 200 nt.

According to a third protocol we transformed with a Promoter-less fulllength aphVIII connected to 720 basepairs of gfp (ss-M13-BZ301) resulting in a 1.4 kb sequence of homology 5′ contiguous to the recipient deletion. In former experiments, promoter-deletion from double-stranded aphVIII caused a 5-140 fold reduction of transformants compared to homologues that were linked to promoters of different strength (Sizova et al. 2001). Promoter-less aphVIII is able to jump in frame into any other gene, the transcription of which is driven by a moderate promoter. gfp-aphVIII was directly cloned into M13mp18 (New England BioLabs) phage (plasmid M13-BZ301). Single-stranded DNA was prepared with according to standard methods. ss DNA was purified on 1% agarose gels in 4×TAE The DNA obtained was digested with SacII to remove residual ds-DNA contaminations and run again through 1% agarose in 4×TAE. After transformation of strain T60-9 with 30 μg DNA 30 transformants appeared. Clones were analysed accordin to the second protocol. 4 clones were homologous recombinants. Two were analyzed by DNA blotting. Both showed single integration by double cross over and repair of the aphVIII gene. By comparing the number of clones that had appeared after transformation with the single-stranded M13-BZ301 vector and double-stranded replicative form, the number of non-homologous recombinants is reduced about 300 times with promoter-less constructs. With promoter-less constructs only recombinations that occurred in frame into an active exon become visible as a clone.

### 4. Disruption of the Endogenous Chlamydomonas Gene: Chop1/Cop3

Disruption of endogenous genes seemed to be more difficult compared to a test gene because the test gene preferentially integrates into an area of the genome that is actively transcribed. Moreover, it contains a strong promoter that keeps the DNA region open for transcription most of the time during cell cycle. In contrast, most endogenous genes possess weak promoters and are active only during defined time windows of the life cycle. We have inactivated the channelopsin-1 gene (GeneBank Accession No: AF508967) which encodes a directly light-gated ion channel (Nagel et al. 2002 Science 296,2395-2398, Sineshchekov et al. 2002 PNAS 99,8689-8694). Two chop1-gene fragments (nucleotide 262 to 3127) and a 1,4 kb-fragment of chop1-gene (4978 to 6361) were inserted adjacent to the functional aphVIII-gene (selection marker with promoter) (FIG. 3f, SEQ ID NO: 6) Finally we produce ssDNA by linear PCR reaction using the primer: chop1-1 (SEQ ID NO: 13): CACTCTTGAGAACAATGGTTCTGT.

Chop1-disruption protocol: For selection of clones with a disrupted chop1 gene (in the data base named CSOA encoding channelopsin-1, GneBank Accession No: AF508967) the aphVIII gene was used as positive selection marker. Two chop1 gene fragments, one of 3 kb DNA (nucleotide 262 to 3127) and one of 1,4 kb- (4978 to 6361) were produced by PCR primers (chop1-2 (SEQ ID NO: 14): aaaagcggccgcCACTCTTGAGAACAATGGTTCTGT, chop1-3 (SEQ ID NO: 15): aaaatctagaTCGGTCCATTGCTCTCTGCTAC, chop1-4 (SEQ ID NO: 16) :aaaaggtaccGCTCTGCGCCCTCTCCGCTG, chop1-5 (SEQ ID NO: 17): aaaaagaagagcAAGCCAAAGCCGTTCCATCCAG, lower case letters define non-Chop1-restriction sites). PCR-products were inserted at 5′ and 3′ ends of aphVIII gene marker (by Xba I, Not I at the 5′ end and Kpn I, Sap I at the 3′ end).

The ssDNA were produced by linear PCR reaction from primer: chop1-1: CACTCTTGAGAACAATGGTTCTGT. 35 circles have been used per reaction, 60° C. for primer annealing and 6 min at 72° C. for primer extension. A total PCR product was purified with the NucleoSpin Plasmid Kit (Macherey-Nagel, Cat. No. 740 588.250). In order to cleave the double stranded template DNA purified PCR products were incubated with Dpn I and Sac II endonucleases (NEB, Frankfurt, Germany). The ssDNA thus obtained was used for transformation (10 μg per transformation) of C. reinhardtii, strain Cw2, according to the standard PEG-glass beads procedure. Transformants that had survived on 20 mg/l paromomycin were grown in low light up to OD_{800 nm}=0.2, harvested, and the level of Chop1-protein was analyzed by protein gel blotting and immunodetection (Western blotting). For detection, antisera against Chop1 and Chop2 (Channelopsin-2) were used. For the identification of clones with disrupted chop1 gene two independent PCRs with two separate pairs of primers was used: One reaction with Chop1del-w (SEQ ID NO: 18): CTGCGACTTCGTCCTCATGCA and Chop1del-rev (SEQ ID NO: 19): ATGCCGCCAGTC-ATGCCGG, to monitor deletion of the middle part in chop1 gene, which should be replaced by the aphVIII marker. This reaction should not produce any homogenous product if chop1 gene was disrupted. In a second reaction with APH-fw (SEQ ID NO: 20): gacagcacagtgtggacgttg and Chop1-end-rev (SEQ ID NO: 21): CTATTGATTGCAGGAGGCGCAG and sequencing of the product it was confirmed that aph marker integrated in to the chop1 gene.

In another preferred protocol again two fragments of chop1: AF508967 were cloned on both sides of aphVIII-gene but the fragment 5′ of aphVIII was cloned in frame with the coding sequence of apHVIII (FIG. 3g, SEQ ID NO: 7). The following primers were used for amplification of the two fragments: (SEQ ID NO: 22: 1021_NOTI_FW aaagcggccgcTCATCGAGTATTTCCATGTG; SEQ ID NO: 23: 2041_MSCI_RW TTTTGGCCACTCGCTATAATGGCAAGGCC) and (SEQ ID NO: 24: 3200_KPNI_FW: aaaggtaccCCAGATCGCCAACTCACCCC; SEQ ID NO 25: 4580_SAPI_RW: GAGGAAGCGGAAGAGCTGGAGGCGCCGCCCATGCCG), respectively.

1. A method for increasing the ratio of homologous to non-homologous recombination of a polynucleotide into a host cell's DNA, wherein the non-homologous recombination of the polynucleotide into the DNA is suppressed by use of a single-stranded DNA, selected from one or more single-stranded DNA capable of homologous recombination with the cell's DNA.

2. The method according to claim 1, wherein the single-stranded DNA is purified with endonucleases or exonucleases to minimize the presence of dsDNA.

3. The method according to claim 1, wherein the single-stranded DNA comprises a nucleic acid sequence corresponding to a nucleic acid sequence of the cell's DNA, but differing from it by deletion, addition, or substitution of at least one nucleotide.

4. The method according to claim 1, wherein the single-stranded DNA comprises 100 to 30,000 nucleotides.

5. The method according to claim 1, wherein the single-stranded DNA further comprises a nucleic acid sequence acting as a selection marker.

6-29. (canceled)

30. The method according to claim 5, wherein the selection marker is constructed in such a way that it can be removed from the host cell.

31. The method according to claim 5, wherein the selection marker codes for resistance to an antibiotic.

32. The method according to claim 31, wherein the selection marker is derived from an aminophosphotransferase gene (aph).

33. The method according to claim 32, wherein the aph gene is aph VIII from Streptomyces rimosus.

34. The method according to claim 1, wherein the method is used for the generation of transformants by transforming a host cell with at least a single-stranded DNA capable of recombining with the cell's DNA.

35. The method according to claim 34, wherein the transformants are selected by use of the selection marker.

36. The method according to claim 35, wherein the selection marker is constructed in such a way that it can be removed from the host cell.

37. The method according to claim 1, wherein the single-stranded DNA does not contain a nucleotide sequence that might serve as an origin of replication.

38. The method according to claim 1, wherein the single-stranded DNA is covered with a single-strand binding protein and transformation is carried out with the resulting DNA/protein filament.

39. The method according to claim 38, wherein the single-strand binding protein is RecA and/or Rad 51, or a homolog thereof.

40. The method according to claim 1, wherein the host cell overexpresses proteins that promote the recombination process.

41. The method according to claim 40, wherein recA and/or rad51 or a homolog thereof are overexpressed.

42. The method according to claim 1, wherein the single-stranded DNA is produced using a single-stranded phage.

43. The method according to claim 42, wherein the phage is M13 or a derivative thereof.

44. The method according to claim 1, wherein the single-stranded DNA is produced via primer extension from a linearized double-stranded plasmid.

45. The method according to claim 1, wherein the single-stranded DNA is generated from a double-stranded fragment by treatment with exonuclease III (Exo III).

46. The method according to claim 1, wherein the method is applied to eukaryotes.

47. The method according to claim 46, wherein the eukaryote is a plant.

48. The method according to claim 47, wherein the plant is a green alga.

49. The method according to claim 48, wherein the green alga is Chlamydomonas rheinhardtii.

50. The method according to claim 1, wherein the method is applied to prokaryotes.

51. Mixture of transformants obtainable by transforming a host cell in the presence of single-stranded DNA selected from one or more single stranded DNA capable of recombining with the cell's DNA.

52. Mixture of transformants according to claim 51, wherein the ratio of transformants resulting from homologous and non-homologous recombination events is larger than 1:100.

53. The mixture according to claim 52, wherein the ratio of transformants resulting from homologous and non-homologous recombination events is larger than 1:10.

54. The mixture according to claim 52, wherein the ratio of transformants resulting from homologous and non-homologous recombination events is larger than 1:3.


SEQUENCE LISTING (26 sequences)

SEQ ID NO: 1 - DNA, 2487 bp, Artificial Sequence
  synthetic
cggcggggagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggtacccaagggcgaggagctgttcaccggtgtggtccccatcctggtggagctggacggcgacgtgaacggccacaagttctccgtctccggcgagggtgagggtgacgccacctacggcaagctgaccctgaagttcatctgcaccaccggcaagctgcccgtgccctggcccaccctggtcaccaccctgacctacggtgtgcagtgcttctcccgctaccccgaccacatgaagcagcacgacttcttcaagtccgccatgcccgagggctacgtgcaggagcgcaccatcttcttcaaggacgacggcaactacaagacccgcgccgaggtcaagttcgagggcgacaccctggtgaaccgcatcgagctgaagggcatcgacttcaaggaggacggcaacatcctgggccacaagctggagtacaactacaactcccacaacgtgtacatcatggccgacaagcagaagaacggcatcaaggtgaacttcaagatccgccacaacatcgaggacggctccgtgcagctggccgaccactaccagcagaacacccccatcggcgatggccccgtgctgctgcccgacaaccactacctgtccatccagtccgccctgtccaaggaccccaacgagaagcgcgaccacatggtcctgctggagttcgtcaccgctgccggcatcacccacggcatggacgagctgtacaagctcgagattcgaagcatggacgatgcgttgcgtgcactgcggggtcggtatcccggttgtgagtgggttgttgtggaggatggggcctcgggggctggtgtttatcggcttcggggtggtgggcgggagttgtttgtcaaggtggcagctctgggggccggggtgggcttgttgggtgaggctgagcggctggtgtggttggcggaggtggggattcccgtacctcgtgttgtggagggtggtggggacgagagggtcgcctggttggtcaccgaagcggttccggggcgtccggccagtgcgcggtggccgcgggagcagcggctggacgtggcggtggcgctcgcggggctcgctcgttcgctgcacgcgctggactgggagcggtgtccgttcgatcgcagtctcgcggtgacggtgccgcaggcggcccgtgctgtcgctgaagggagcgtcgacttggaggatctggacgaggagcggaaggggtggtcgggggagcggcttctcgccgagctggagcggactcggcctgcggacgaggatctggcggtttgccacggtgacctgtgcccggacaacgtgctgctcgaccctcgtacctgcgaggtgaccgggctgatcgacgtggggcgggtcggccgctctacaactagtggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcg

SEQ ID NO: 2 - DNA, 2662 bp, Artificial Sequence
  synthetic
cggcggggagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggtacccaagggcgaggagctgttcaccggtgtggtccccatcctggtggagctggacggcgacgtgaacggccacaagttctccgtctccggcgagggtgagggtgacgccacctacggcaagctgaccctgaagttcatctgcaccaccggcaagctgcccgtgccctggcccaccctggtcaccaccctgacctacggtgtgcagtgcttctcccgctaccccgaccacatgaagcagcacgacttcttcaagtccgccatgcccgagggctacgtgcaggagcgcaccatcttcttcaaggacgacggcaactacaagacccgcgccgaggtcaagttcgagggcgacaccctggtgaaccgcatcgagctgaagggcatcgacttcaaggaggacggcaacatcctgggccacaagctggagtacaactacaactcccacaacgtgtacatcatggccgacaagcagaagaacggcatcaaggtgaacttcaagatccgccacaacatcgaggacggctccgtgcagctggccgaccactaccagcagaacacccccatcggcgatggccccgtgctgctgcccgacaaccactacctgtccatccagtccgccctgtccaaggaccccaacgagaagcgcgaccacatggtcctgctggagttcgtcaccgctgccggcatcacccacggcatggacgagctgtacaagctcgagattcgaagcatggacgatgcgttgcgtgcactgcggggtcggtatcccggttgtgagtgggttgttgtggaggatggggcctcgggggctggtgtttatcggcttcggggtggtgggcgggagttgtttgtcaaggtggcagctctgggggccggggtgggcttgttgggtgaggctgagcggctggtgtggttggcggaggtggggattcccgtacctcgtgttgtggagggtggtggggacgagagggtcgcctggttggtcaccgaagcggttccggggcgtccggccagtgcgcggtggccgcgggagcagcggctggacgtggcggtggcgctcgcggggctcgctcgttcgctgcacgcgctggactgggagcggtgtccgttcgatcgcagtctcgcggtgacggtgccgcaggcggcccgtgctgtcgctgaagggagcgtcgacttggaggatctggacgaggagcggaaggggtggtcgggggagcggcttctcgccgagctggagcggactcggcctgcggacgaggatctggcggtttgccacggtgacctgtgcccggacaacgtgctgctcgaccctcgtacctgcgaggtgaccgggctgatcgacgtggggcgggtcggccgtgcggaccggcactccgatctcgcgctggtgctgcgcgagctggcccacgaggaggacccgtggttcgggccggagtgttccgcggcgttcctgcgggagtacgggcgcgggtgggatggggcggtatcggaggaaaagctggcgttttaccggctgttggacgagttcttctgactctacaactagtggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcg

SEQ ID NO: 3 - DNA, 1564 bp, Artificial Sequence
  synthetic
cggcggggagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggacgatgcgttgcgtgcactgcggggtcggtatcccggttgtgagtgggttgttgtggaggatggggcctcgggggctggtgtttatcggcttcggggtggtgggcgggagttgtttgtcaaggtggcagctctgggggccggggtgggcttgttgggtgaggctgagcggctggtgtggttggcggaggtggggattcccgtacctcgtgttgtggagggtggtggggacgagagggtcgcctggttggtcaccgaagcggttccggggcgtccggccagtgcgcggtggccgcgggagcagcggctggacgtggcggtggcgctcgcggggctcgctcgttcgctgcacgcgctggactgggagcggtgtccgttcgatcgcagtctcgcggtgacggtgccgcaggcggcccgtgctgtcgctgaagggagcgtcgacttggaggatctggacgaggagcggaaggggtggtcgggggagcggcttctcgccgagctggagcggactcggcctgcggacgaggatctggcggtttgccacggtgacctgtgcccggacaacgtgctgctcgaccctcgtacctgcgaggtgaccgggctgatcgacgtggggcgggtcggccgtgcggaccggcactccgatctcgcgctggtgctgcgcgagctggcccacgaggaggacccgtggttcgggccggagtgttccgcggcgttcctgcgggagtacgggcgcgggtgggatggggcggtatcggaggaaaagctggcgttttaccggctgttggacgagttcttctgactctacaactagtggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcg

SEQ ID NO: 4 - DNA, 917 bp, Artificial Sequence
  synthetic
gtcaaggtggcagctctgggggccggggtgggcttgttgggtgaggctgagcggctggtgtggttggcggaggtggggattcccgtacctcgtgttgtggagggtggtggggacgagagggtcgcctggttggtcaccgaagcggttccggggcgtccggccagtgcgcggtggccgcgggagcagcggctggacgtggcggtggcgctcgcggggctcgctcgttcgctgcacgcgctggactgggagcggtgtccgttcgatcgcagtctcgcggtgacggtgccgcaggcggcccgtgctgtcgctgaagggagcgtcgacttggaggatctggacgaggagcggaaggggtggtcgggggagcggcttctcgccgagctggagcggactcggcctgcggacgaggatctggcggtttgccacggtgacctgtgcccggacaacgtgctgctcgaccctcgtacctgcgaggtgaccgggctgatcgacgtggggcgggtcggccgtgcggaccggcactccgatctcgcgctggtgctgcgcgagctggcccacgaggaggacccgtggttcgggccggagtgttccgcggcgttcctgcgggagtacgggcgcgggtgggatggggcggtatcggaggaaaagctggcgttttaccggctgttggacgagttcttctgacgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcg

SEQ ID NO: 5 - DNA, 4859 bp, Artificial Sequence
  synthetic
cggcggggagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatggccatgggtgccgacgacgtcgtggactcgtccaagtcgttcgtcatggagaacttctcctcctaccacgggaccaagcccggttacgtggactcgatccagaagggaatccagaagcccaagtcgggcacccaggggaactacgacgacgactggaagggcttctactccaccgacaacaagtacgacgccgccggttactcggtggacaacgagaaccccctgtcgggcaaggccgggggtgtcgtcaaggtgacctaccccggactcaccaaggtgctggccctgaaggtcgacaatgctgagaccattaagaaggagctgggactgtccctgaccgagcccctgatggagcaggtcggaaccgaggagttcatcaagcgcttcggtgatggagcctcccgcgtggtcctgtcgctgcccttcgccgagggatcgtcctccgtggagtacatcaacaactgggagcaggccaaggccctgagcgtggagctggagatcaacttcgagactcgcggaaagcgcggacaggatgccatgtacgagtacatggcccaggcctgcgccggtaaccgcgtccgccgcattatgtaatgaggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcggcggccgccggcggggagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggacgatgcgttgcgtgcactgcggggtcggtatcccggttgtgagtgggttgttgtggaggatggggcctcgggggctggtgtttatcggcttcggggtggtgggcgggagttgtttgtcaaggtggcagctctgggggccggggtgggcttgttgggtgaggctgagcggctggtgtggttggcggaggtggggattcccgtacctcgtgttgtggagggtggtggggacgagagggtcgcctggttggtcaccgaagcggttccggggcgtccggccagtgcgcggtggccgcgggagcagcggctggacgtggcggtggcgctcgcggggctcgctcgttcgctgcacgcgctggactgggagcggtgtccgttcgatcgcagtctcgcggtgacggtgccgcaggcggcccgtgctgtcgctgaagggagcgtcgacttggaggatctggacgaggagcggaaggggtggtcgggggagcggcttctcgccgagctggagcggactcggcctgcggacgaggatctggcggtttgccacggtgacctgtgcccggacaacgtgctgctcgaccctcgtacctgcgaggtgaccgggctgatcgacgtggggcgggtcggccgtgcggaccggcactccgatctcgcgctggtgctgcgcgagctggcccacgaggaggacccgtggttcgggccggagtgttccgcggcgttcctgcgggagtacgggcgcgggtgggatggggcggtatcggaggaaaagctggcgttttaccggctgttggacgagttcttctgactctacaactagtggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgccggcggggagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatggccatgggtgccgacgacgtcgtggactcgtccaagtcgttcgtcatggagaacttctcctcctaccacgggaccaagcccggttacgtggactcgatccagaagggaatccagaagcccaagtcgggcacccaggggaactacgacgacgactggaagggcttctactccaccgacaacaagtacgacgccgccggttactcggtggacaacgagaaccccctgtcgggcaaggccgggggtgtcgtcaaggtgacctaccccggactcaccaaggtgctggccctgaaggtcgacaatgctgagaccattaagaaggagctgggactgtccctgaccgagcccctgatggagcaggtcggaaccgaggagttcatcaagcgcttcggtgatggagcctcccgcgtggtcctgtcgctgcccttcgccgagggatcgtcctccgtggagtacatcaacaactgggagcaggccaaggccctgagcgtggagctggagatcaacttcgagactcgcggaaagcgcggacaggatgccatgtacgagtacatggcccaggcctgcgccggtaaccgcgtccgccgcattatgtaatgaggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcg

SEQ ID NO: 6 - DNA, 6107 bp, Artificial Sequence
  synthetic
tgagaacaatggttctgttatttgcatcccgaacaacggccagtgcttctgcttggtgagtcgggcgcgcccgccctgccgtcctgtgcgcgttttgaggtagtagcctgttttgagacttgatttacttccattactggattgcaccgcttgctatggacggcagggttcagatactgaggcgagtgctcacatgtgtgccttgccctgacacccacaggcttggcttaaatccaacggaacaaatgccgagaagttggctgccaacattctgcagtgggtgagttgtggggccactgaaccttttccgacagcgacttcgcaccgtgtcgcggtcggccgcgtctctcttccagctcttgctgttgtccatgtttttctatcacgacattttaaggcgctcggtcatgtttgcggcagcagcgactggccgcgacccctcagtaacctcgcacatctcagctcgcagtggctcccaccccctgcgtccccctgctcatcgtgtctcctgtaaaggtccgctgatgatacccgaatcaaacctttcgcagattacttttgcgctttcagcgctctgcctgatgttctacggctaccagacctggaagtctacttgcggctgggaggagatttacgtggccacgatcgaggtaaggaccccacggaagccgtgcagcgctgcctctgtctatttgcgcaccttcgcatctaaaacaagaattcgtgtgttgcaactgcagatgatcaagttcatcatcgagtatttccatgtgagcagctggtgtgaggcaagcctgacaacgctgtgtgcaggcctaggcacgcgacgcgtccaccggaccccaatacactcaagcccgagtaccagccagggcaggggtgttgcaaaagccgtcatcttaaggtgtgatcgaccgtgccgtggaagctacacaacatgttgggttttgcgatagacaccaaccctgcttgtgccgggcatcgtggcttgaccctgtcctggcaatctactcctggcgcggttcacgcccacaggagtttgacgaacctgcggtgatctactcatccaacggcaacaagaccgtgtggcttcgttacgcggagtggctgctgacctgcccgtgagtctgacttccgcagacggccctgtcgcctgtcccttcagcgctgtgtagcctggcgcgattcgcgcttaaagcatttgctgacaccatgctccccgccccccgcgccccgtacacatcaatccctctccctaccctgtatcgtcctcgtcaccgctctggccctgcatgcaactgacccacagcgtgagttgtcggcgggtgcggaatgcttgcacgaggatggcggccgtggacacaagggatccctgcaatccggcagccgcactggaaggcccattgcataacctgtatcgaaccgcccaaacctatcccaaccacactctttaacgcccgcagtgtcattcttatccatctgagcaaccttacgggtctggcgaacgactataacaagcgtaccatgggtctgctggtgtcagagtgagtctgtgggggtggggggatgccaggggagctgtggcgcgcgtctggggatcagacgatgagaaacgctggaagcggcaggaagcgccgcgcgggcactggcgtgagggcactggcgctcaccggtcttgcagtgcggctatgcaaggcagcatgcggcggtggtgtgcgtccgagccgcccccgcctatcgcataccgtgctgcggccttgccattatagcgaggccatgccgtgaaccctttttcctcctgctcctcctcctgctaattctgccgctgcagtatcggcacgatcgtgtggggcaccacggccgcgctgtccaagggatacgtccgtgtcattttcttcctgatgggcctgtgctacggcatctacacattcttcaacgcagccaaggtgcggggcgtgctgttgcttggcatgtttgcgagctcgtgcgaagtatcatcgtcaaccagcagcccggtgtcgccgcagcgccatgcagcgacaacccaatgtcccacaatgcatgatctgaccggggaccgcctgagcgtgtacggaaccgctgacgggtgtgctcgtgcctgtactgtgtgtccccctccccccaacgtgtgccttgtgggcggcaggtctacattgaggcgtaccacaccgtgcccaagggcatttgccgcgacctggtccgctaccttgcctggctctacttctgttcatggtgcgtttggtcctggtgggggttataattcctggcctggtctgggaggatgagcaatcacagacagcgtctgacatgggatggggggagttgaaagctgggcgatcaggtgcagtctgaggtccggcattcgatgcggtccatactgcttcagctgacttgtcaccccgcacttgtggactgctcagggctatgttcccggtgctgttcctgctgggccccgagggctttggccacatcaaccaattcagtgagtccacgtgttgtggtggtgtgtgtgtggggggggggggagtctccatgcccccgggtgtgctggcgtccatggagtgtaagcccatcgtggggtcgtgggcgacaggcttggagtactgcaacctccaacgggaatgcaatgggcacgttgttgctccatcagcgctgtttggttcctggcgctataacgctgcgggtatgtcgtttgctgcagactctgccatcgcccacgccatcctggaccttgcctccaagaacgcttggagtatgatgggtcactttctgcgtgtcaaggtgcgaggggcggcgggcgggggacctgcaaagacagagtgtaaattcacaggggcggtagcaggcggccgccggcggggagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggacgatgcgttgcgtgcactgcggggtcggtatcccggttgtgagtgggttgttgtggaggatggggcctcgggggctggtgtttatcggcttcggggtggtgggcgggagttgtttgtcaaggtggcagctctgggggccggggtgggcttgttgggtgaggctgagcggctggtgtggttggcggaggtggggattcccgtacctcgtgttgtggagggtggtggggacgagagggtcgcctggttggtcaccgaagcggttccggggcgtccggccagtgcgcggtggccgcgggagcagcggctggacgtggcggtggcgctcgcggggctcgctcgttcgctgcacgcgctggactgggagcggtgtccgttcgatcgcagtctcgcggtgacggtgccgcaggcggcccgtgctgtcgctgaagggagcgtcgacttggaggatctggacgaggagcggaaggggtggtcgggggagcggcttctcgccgagctggagcggactcggcctgcggacgaggatctggcggtttgccacggtgacctgtgcccggacaacgtgctgctcgaccctcgtacctgcgaggtgaccgggctgatcgacgtggggcgggtcggccgtgcggaccggcactccgatctcgcgctggtgctgcgcgagctggcccacgaggaggacccgtggttcgggccggagtgttccgcggcgttcctgcgggagtacgggcgcgggtgggatggggcggtatcggaggaaaagctggcgttttaccggctgttggacgagttcttctgactctacaactagtggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgccgccctctccgctgagctcgcagcccggcatcagccctggcatggcgacgccgcccgccgccaccgccgcacccgccgctggcggcagcgaggccgagatgctgcagcagctgatgagcgaggtgcgtgtagcagcagggctgtgttggcgccggctggcgtttcaagggagggtcagggaaggcggtagtctggcaaccgggagttttcggcgatgggattatgggccagaaacagaccggaatggcgtcgttgaaagcacccgcgtcccgacggcgtccgtcggtctgctgtggctgtggcctgttgctgacggctgctgggcctcgtgctgttgtattgcagatcaaccgcctgaagaacgagctgggcgagtaaactgctggcccagccgtacggacatatgcctgctgaggcaccagcgccgcaacacacatcgccgcagctgtcgcggctgccatgttggatttgcgcgtggcggcgtggtggtgtggtggtgtggtggcaggaacaagggcgaagctttaacttacccggcgctcagcgcttcgttcataggttcggcgcttgagccgtggtagcggcaagtgtgcccgcggcaacgcggggcaaagcgaagacgccgatgacttgacgcctggtatgacaccttggtctatgaagtcgcgctgcggtgctgggatcaagaaacagcaactcgaggaaggtatcatcgagcgtcgttatacagcagacaaggtacgaaacggtgtgcaggagggcatgcacagcagcttcaaatggcacgtgcatggctctgttgcgaacaagctgctctgagacacggattgagagcccttaatcggtggtcacaagaggtggggttacggtatcggggcgctgcgatagtcctgcaagtgctgcctgttgaacacaagggctcagaatttatggcagggaaggtcaaggccgagaatggccgcgtgcgtgatttattgtttgagccagggcttgttgatactgtattaatcatgcgtgtgtgtttgtgtgcgtgaacgtgacccgacggattccgtgagccgctgcgcatgcaagatccggccctgacctatgtcctagtacaagccgatcgtgcttggcctgccttgattaatgcgtcgcctgaggattcccgtttgtggcttttaaggagcgcgaatacggcagttacgtgacctgcttgtcgggttggggaaatccgtctggtgtgtacctggcctggccggctgatcgggtctgcttccggcaagtaactgtgcgggtgaaactacaaaaggcagcgccggttgtgggcgtcgttttggttggtttggcggggttcccattgcaatgtgtgtttccataaatcatgggcgacactggatgg

SEQ ID NO: 7 - DNA, 3531 bp, Artificial Sequence
  synthetic
gcggccgctcatcgagtatttccatgtgagcagctggtgtgaggcaagcctgacaacgctgtgtgcaggcctaggcacgcgacgcgtccaccggaccccaatacactcaagcccgagtaccagccagggcaggggtgttgcaaaagccgtcatcttaaggtgtgatcgaccgtgccgtggaagctacacaacatgttgggttttgcgatagacaccaaccctgcttgtgccgggcatcgtggcttgaccctgtcctggcaatctactcctggcgcggttcacgcccacaggagtttgacgaacctgcggtgatctactcatccaacggcaacaagaccgtgtggcttcgttacgcggagtggctgctgacctgcccgtgagtctgacttccgcagacggccctgtcgcctgtcccttcagcgctgtgtagcctggcgcgattcgcgcttaaagcatttgctgacaccatgctccccgccccccgcgccccgtacacatcaatccctctccctaccctgtatcgtcctcgtcaccgctctggccctgcatgcaactgacccacagcgtgagttgtcggcgggtgcggaatgcttgcacgaggatggcggccgtggacacaagggatccctgcaatccggcagccgcactggaaggcccattgcataacctgtatcgaaccgcccaaacctatcccaaccacactctttaacgcccgcagtgtcattcttatccatctgagcaaccttacgggtctggcgaacgactataacaagcgtaccatgggtctgctggtgtcagagtgagtctgtgggggtggggggatgccaggggagctgtggcgcgcgtctggggatcagacgatgagaaacgctggaagcggcaggaagcgccgcgcgggcactggcgtgagggcactggcgctcaccggtcttgcagtgcggctatgcaaggcagcatgcggcggtggtgtgcgtccgagccgcccccgcctatcgcataccgtgctgcggccttgccattatagcgaggccatgccgtgaaccctttttcctcctgctcctcctcctgctaattctgccgctgcagtatcggcacgatggccatggacgatgcgttgcgtgcactgcggggtcggtatcccggttgtgagtgggttgttgtggaggatggggcctcgggggctggtgtttatcggcttcggggtggtgggcgggagttgtttgtcaaggtggcagctctgggggccggggtgggcttgttgggtgaggctgagcggctggtgtggttggcggaggtggggattcccgtacctcgtgttgtggagggtggtggggacgagagggtcgcctggttggtcaccgaagcggttccggggcgtccggccagtgcgcggtggccgcgggagcagcggctggacgtggcggtggcgctcgcggggctcgctcgttcgctgcacgcgctggactgggagcggtgtccgttcgatcgcagtctcgcggtgacggtgccgcaggcggcccgtgctgtcgctgaagggagcgtcgacttggaggatctggacgaggagcggaaggggtggtcgggggagcggcttctcgccgagctggagcggactcggcctgcggacgaggatctggcggtttgccacggtgacctgtgcccggacaacgtgctgctcgaccctcgtacctgcgaggtgaccgggctgatcgacgtggggcgggtcggccgtgcggaccggcactccgatctcgcgctggtgctgcgcgagctggcccacgaggaggacccgtggttcgggccggagtgttccgcggcgttcctgcgggagtacgggcgcgggtgggatggggcggtatcggaggaaaagctggcgttttaccggctgttggacgagttcttctgactctacaactagtggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgggtaccgagcacatcctgctgtacggcgacatccgcaagaagcagaaggtcaacgtggctggccaggagatggaggtggagaccatggtgcacgaggaggacgacgagacgcagaaggtgcccacggcaaagtacgccaaccgcgactcgttcatcatcatgcgcgaccgcctcaaggagaagggcttcgagacccgcgcctcgctggacggcgacccgaacggcgacgccgaggccaacgctgcagccggcggcaagcccggaatggagatgggcaagatgaccggcatgggcatgagcatgggtgccggcatgggcatggcgaacatcgattcgggccgcgtcatcctcgccgtgccggacgtgagtgatgtgctgtcgcggggcggtgcggggcggggctagcgcgtgggggcttgaacaggaaagagcagcagccgcggagtatgctaagcagaaagaggcgactcttggcgcacgtgtgtgcgccggccttcagacaccaagtcgtctgatgtccgcgcgtgccctcctcaccgcgtcacccacgcacggtgtcactccacccaccctacgcctggccaccccttaccctcaccatctccttcagatctccatggtggactttttccgcgagcagttcgcgcggctgcccgtgccctacgaactggtgcccgcgctgggcgcggagaacaccctccagctggtgcagcaggcgcagtcactgggaggctgcgacttcgtcctcatgcaccccgagttcctgcgcgaccgcagtcccacgggtctgctgccccgcctcaagatgggcgggcagcgcgccgcggccttcggctgggcggcaatcggccccatgcgggacttgatcgagggttcgggcgttgacggctggctggagggccccagctttggcgccggcatcaaccagcaggcgctggtggcgctgatcaaccgcatgcagcaggccaagaagatgggcatgatgggcggtgagtcaggggggggggctgtggaggggtggggtcgcggtggggcggaggcgaggtggaggtgcaggttttggctgtgacggaaggtgttgtgggggcctgtgcagcactgcggaaaccatgggaagaaccaggggaggagcatgcaagctgcgggagagagcctacggtaggaagcagcggccttttcttggaacttgttgcgaacctgcatgtcacccacctctaccctcttgccctcatctttctttccaaacaggtatgggtatgggcatgggcggcggcatgggtatgggcatgggtatgggcatgggcatggcccccagcatgaacgccggcatgactggcggcatgggcggcgcctccagctcttccgcttcctc

SEQ ID NO: 8 - DNA, 19 bp, Artificial Sequence
  synthetic
tggagctccaccgcggtgg

SEQ ID NO: 9 - DNA, 20 bp, Artificial Sequence
  synthetic
tgggtacccgcttcaaatac

SEQ ID NO: 10 - DNA, 21 bp, Artificial Sequence
  synthetic
gagatcggcgagcagccgtgg

SEQ ID NO: 11 - DNA, 21 bp, Artificial Sequence
  synthetic
gagcagtatcttccatccacc

SEQ ID NO: 12 - DNA, 21 bp, Artificial Sequence
  synthetic
accagcgcgagatcggagtgc

SEQ ID NO: 13 - DNA, 24 bp, Artificial Sequence
  synthetic
cactcttgagaacaatggttctgt

SEQ ID NO: 14 - DNA, 36 bp, Artificial Sequence
  synthetic
aaaagcggccgccactcttgagaacaatggttctgt

SEQ ID NO: 15 - DNA, 32 bp, Artificial Sequence
  synthetic
aaaatctagatcggtccattgctctctgctac

SEQ ID NO: 16 - DNA, 30 bp, Artificial Sequence
  synthetic
aaaaggtaccgctctgcgccctctccgctg

SEQ ID NO: 17 - DNA, 34 bp, Artificial Sequence
  synthetic
aaaaagaagagcaagccaaagccgttccatccag

SEQ ID NO: 18 - DNA, 21 bp, Artificial Sequence
  synthetic
ctgcgacttcgtcctcatgca

SEQ ID NO: 19 - DNA, 19 bp, Artificial Sequence
  synthetic
atgccgccagtcatgccgg

SEQ ID NO: 20 - DNA, 21 bp, Artificial Sequence
  synthetic
gacagcacagtgtggacgttg

SEQ ID NO: 21 - DNA, 22 bp, Artificial Sequence
  synthetic
ctattgattgcaggaggcgcag

SEQ ID NO: 22 - DNA, 31 bp, Artificial Sequence
  synthetic
aaagcggccgctcatcgagtatttccatgtg

SEQ ID NO: 23 - DNA, 29 bp, Artificial Sequence
  synthetic
ttttggccaatcgtgccgatactgcagcg

SEQ ID NO: 24 - DNA, 29 bp, Artificial Sequence
  synthetic
aaaggtaccgagcacatcctgctgtacgg

SEQ ID NO: 25 - DNA, 36 bp, Artificial Sequence
  synthetic
gaggaagcggaagagctggaggcgccgcccatgccg

SEQ ID NO: 26 - DNA, 2791 bp, Artificial Sequence
  synthetic
gagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaagtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatggccaagctgaccagcgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggtacccaagggcgaggagctgttcaccggtgtggtccccatcctggtggagctggacggcgacgtgaacggccacaagttctccgtctccggcgagggtgagggtgacgccacctacggcaagctgaccctgaagttcatctgcaccaccggcaagctgcccgtgccctggcccaccctggtcaccaccctgacctacggtgtgcagtgcttctcccgctaccccgaccacatgaagcagcacgacttcttcaagtccgccatgcccgagggctacgtgcaggagcgcaccatcttcttcaaggacgacggcaactacaagacccgcgccgaggtcaagttcgagggcgacaccctggtgaaccgcatcgagctgaagggcatcgacttcaaggaggacggcaacatcctgggccacaagctggagtacaactacaactcccacaacgtgtacatcatggccgacaagcagaagaacggcatcaaggtgaacttcaagatccgccacaacatcgaggacggctccgtgcagctggccgaccactaccagcagaacacccccatcggcgatggccccgtgctgctgcccgacaaccactacctgtccatccagtccgccctgtccaaggaccccaacgagaagcgcgaccacatggtcctgctggagttcgtcaccgctgccggcatcacccacggcatggacgagctgtacaagctcgagattcgaagcatggacgatgcgttgcgtgcactgcggggtcggtatcccggttgtgagtgggttgttgtggaggatggggcctcgggggctggtgtttatcggcttcggggtggtgggcgggagttgtttgtcaaggtggcagctctgggggccggggtgggcttgttgggtgaggctgagcggctggtgtggttggcggaggtggggattcccgtacctcgtgttgtggagggtggtggggacgagagggtcgcctggttggtcaccgaagcggttccggggcgtccggccagtgcgcggtggccgcgggagcagcggctggacgtggcggtggcgctcgcggggctcgctcgttcgctgcacgcgctggactgggagcggtgtccgttcgatcgcagtctcgcggtgacggtgccgcaggcggcccgtgctgtcgctgaagggagcgtcgacttggaggatctggacgaggagcggaaggggtggtcgggggagcggcttctcgccgagctggagcggactcggcctgcggacgaggatctggcggtttgccacggtcacctgtgcccggacaacgtgctgctcgaccctcgtacctgcgaggtgaccgggctgatcgacgtggggcgggtcggccgctctacaactagtggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgggtacc